(19) United States
(12) Patent Application Publication
(10) Pub. No.: US 2008/0181857 A1
(43) Pub. Date: Jul. 31, 2008

(54) Dental flosses incorporating active agents

(75) Inventors: Ruben Eliam Avila, Mason, OH (US); Daniel William Volpenhein, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company, Cincinnati, OH (US)

(21) Appl. No.: 12/009,655

(22) Filed: Jan. 22, 2008

Related U.S. Application Data
(60) Provisional application No. 60/897,624, filed on Jan. 26, 2007.

(51) Int. Cl. A61C 15/04; A61K 8/19

(52) U.S. Cl. 424/57

(57) ABSTRACT

Disclosed is a dental floss formed from at least one fiber and incorporating a polyphosphate salt.

### CROSS REFERENCE TO RELATED APPLICATION

This application claims the benefit of U.S. Provisional Application No. 60/897,624 filed Jan. 26, 2007, the substances of which are incorporated herein by reference.

### TECHNICAL FIELD

The present invention relates to dental flosses and oral care methods for treating and protecting teeth against erosion.

### BACKGROUND OF THE INVENTION

Oral care products, such as toothpastes and dental flosses, are routinely used by consumers as part of their oral care hygiene regimens. There is a desire to provide additional benefits to teeth surfaces during an oral care session.

### SUMMARY OF THE INVENTION

The invention includes dental flosses incorporating one or more erosion control agents.

### DESCRIPTION OF THE DRAWINGS

The present invention may be understood by reference to the accompanying drawings, in which:

FIG. 1 is a perspective view of one dental floss fiber of the present invention; and

FIG. 2 is schematic representation of the dental floss fiber of FIG. 1 compressing as it passes between a plurality of teeth.

### DESCRIPTION OF THE INVENTION

The following text sets forth a broad description of numerous different embodiments of the present invention. The description is to be construed as exemplary only and does not describe every possible embodiment since describing every possible embodiment would be impractical, if not impossible, and it will be understood that any feature, characteristic, component, composition, ingredient, dosage, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims. All publications and patents cited herein are incorporated herein by reference. All documents cited are incorporated herein by reference; the citation of any document is not to be construed as an admission that it is prior art with respect to the present invention.

Herein, “comprising” means that other steps and other ingredients which do not affect the end result can be added. This term encompasses the terms “consisting of” and “consisting essentially of”.

The term “floss” is intended to encompass a thread-like or fiber-like material suitable for use in cleaning between teeth.

The term “teeth”, as used herein, refers to natural teeth as well as artificial teeth or dental prosthesis.

The term “orally acceptable carrier” as used herein means any safe and effective materials for use in the compositions of the present invention. Such materials include fluoride ion sources, additional anticalculus agents, buffers, abrasive polishing materials, peroxide sources, alkali metal bicarbonate salts, thickening materials, humectants, water, surfactants, titanium dioxide, flavor system, sweetening agents, xylitol, coloring agents, and mixtures thereof.

Herein, the terms “tartar” and “calculus” are used interchangeably and refer to mineralized dental plaque deposits.

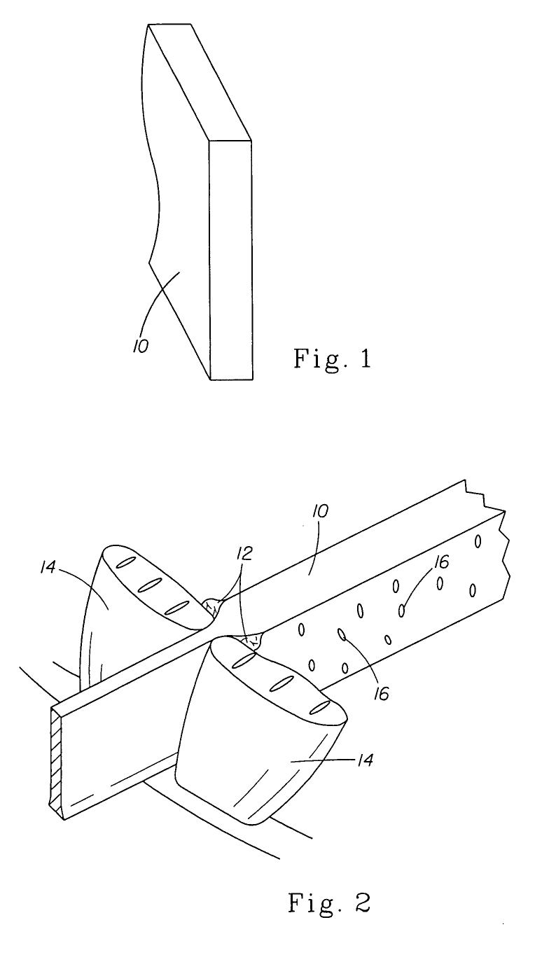

The present invention relates to dental flosses containing erosion control agents, such as polymeric mineral surface active agents, metal ions selected from stannous, zinc and copper, and combinations thereof, which may provide effective protection against dental erosion derived from the deposition on the tooth surface, or portions thereof, of a protective layer or coating comprised of the polymeric mineral surface active agent and/or a highly insoluble film or precipitate of compounds or complexes formed from the reaction of the metal ions with other ingredients of an oral composition and/or components of the enamel surface. The present invention also relates to methods of use of the dental floss, including uses in combination with other oral care compositions which may comprise the same or similar active agents. The dental flosses and methods of the present invention can deposit the erosion control agents, or compositions 12 containing the agents, to tooth surfaces, such as interproximal tooth locations between a plurality of teeth 14, as shown in FIGS. 1 and 2. The erosion control agents can provide a localized benefit at the point of deposition and/or provide a benefit to tooth surfaces distant from the location of deposition. In the later instance, the deposits may act as an agent reservoir for the oral cavity in general, wherein the erosion control agents are solubilized from the deposits within the oral cavity and circulated for a period of time following deposition composition containing the erosion agent from the dental floss.

A variety of dental flosses may be used with the present invention. In one embodiment, a floss, which is described in U.S. Pat. No. 5,518,012, can comprise a single strand of expanded polytetrafluoroethylene (PTFE) fiber that is generally rectangular to oblong in cross-sectional dimensions, as is shown in FIGS. 1 and 2 with respect to floss 10. Typical dimensions may be about 0.02, 0.04, 0.06, 0.08, 0.1 or 0.15 mm to about 2, 1, 0.5, or 0.25 in thickness and about 0.5, 0.7, 1, 1.3, or 1.6 mm to about 5, 4, 3, 2, or 1.5 mm in width. In addition to a monofilament, a dental floss comprising a plurality of filaments (or other forms) might also be used. The filaments may be provided in a variety of cross-sectional shapes, including elliptical, circular, rectangular, square, etc. Where the dental floss is provided in a generally circular shape in cross-section, the diameter may be between about 0.1 mm and about 5 mm, and the dental floss may comprise between about 2 and about 1000 strands. The dental floss may have a closed, open, or expanded structure.

In the example of a monofilament, the dental floss fiber can be produced through a series of processing steps. First, an expanded PTFE sheet is acquired or formed. Such material is available in a variety of forms from a number of commercial sources, such as from W.L. Gore & Associates, Inc., Elkton, Md., under the trademark GORE-TEX®. This material may be formed as taught in U.S. Pat. No. 3,543,566 to Gore. In one embodiment, the sheet may have a thickness of about 0.5 to 1.0 mm; a density of about 0.8 to 1.5 g/cc; and a tenacity of about 0.5 to 1.0 g/tex.

These properties can be measured in a conventional manner. Width and thickness is determined through any conventional means, such as through the use of calipers or through measurements through a scanning electron microscope. Density is determined by dividing the measured weight of the sample by the computed volume of the sample. The volume is computed by multiplying the measured length, width, and thickness of the sample. Tenacity is calculated by dividing the sample's tensile strength by its normalized weight per unit length (tex [grams/1000 meters] or denier [grams/9000 meters]).

This sheet may then be slit into strands by passing the sheet through a series of gapped blades set 0.5 to 20 mm apart. After cutting, the fibers may be subjected to a further heat treatment and/or expansion step, such as through the processes discussed below. Finally, the fibers may be wound onto a spool with care taken to avoid rolling or folding of the fibers during the spooling process.

An expanded PTFE sheet is formed and slit into fibers in the following manner. A fine powder PTFE resin is blended with a lubricant, such as odorless mineral spirits, until a compound is formed. The volume of lubricant used should be sufficient to lubricate the primary particles of the PTFE resin so to minimize the potential of the shearing of the particles prior to extruding.

The compound is then compressed into a billet and extruded, such as through a ram type extruder, to form a coherent extrudate. A reduction ratio of about 30:1 to 300:1 may be used (i.e., reduction ratio=cross-sectional area of extrusion cylinder divided by the cross-sectional area of the extrusion die). For most applications a reduction ratio of 75:1 to 100:1 is preferred.

The lubricant may then be removed, such as through volatilization, and the dry coherent extrudate is expanded in at least one direction about 1.1 to 50 times its original length (with about 1.5 to 2.5 times being preferred). Expansion may be accomplished by passing the dry coherent extrudate over a series of rotating heated rollers or heated plates.

Once this sheet is formed, the sheet may be formed into a fiber by slitting the dry coherent expanded extrudate into predetermined widths by passing it between a set of gapped blades or other cutting means. Following cutting, the slit coherent extrudate may then be further expanded in the longitudinal direction at a ratio of 1:1.1 to 50:1 (with 15:1 to 35:1 being preferred) to form a fiber. Finally, this fiber may be subjected to an amorphous locking step by exposing the fiber to a temperature in excess of 342° C.

The width of the fiber can be controlled by several process variables known in the art of expanding PTFE. Variables which can affect the width of the fiber are: slit width, expansion temperatures and expansion ratio.

Other materials which may be used to from fibers or filaments of dental floss 10 include natural fibers, e.g., cotton and wool, synthetic polymer filaments, e.g., nylon, rayon, polyethylene, polyester, Dacron and acetate polymers, thermoplastic elastomers, e.g., Kratons (e.g., styrene-ethylene or styrene-butylene block copolymers), Pebax (e.g., polyether-polyamide block copolymers), and thermoplastic urethanes. The dental floss may contain a plurality of fibers or filaments as previously discussed. Other dental flosses suitable which may be used with the present invention are also described in U.S. Pat. Nos. 5,518,012; 5,357,990; 5,918,609; 5,937,874; 5,941,256; and 6,026,829.

In one embodiment, the dental flosses of the present invention can comprise a coating or capsule that incorporates the erosion control agent. One example of a capsule 16 (FIG. 2) is a microcapsule having walls that are thin enough to rupture upon the application of a shear force, e.g., the shear force exerted by flossing, yet of sufficient strength to withstand normally applied pressure occurring during manufacturing, handling and packaging of the dental hygiene article. Preferred microcapsules shear under normal flossing forces. Upon rupture of the microcapsule, the agents of the present invention can be released to the interstitial spaces or along, at, or below the gingival margin or any other location where the dental floss is used.

A variety of materials can be used to form the encapsulating wall of a microcapsule, provided it is suitable for application in the mouth. Materials capable of forming microcapsules include, but are not limited to, the following: starch; dextrin; gelatin; gum arabic; casein; paraffin wax; natural waxes such as carnauba wax, beeswax, candelilla wax, Japan wax; styrene maleic acid; polyethylene-ethyl cellulose mixtures; cellulose acetophthalate; polymerized acrylonitrile; butadiene and styrene polymers; acetal copolymers and homopolymers; acrylic resins; allylic resins; amino resins; cellulosic resins; epoxy resins; fluoroplastic resins; Furan polymers; ionomer resins; nitrile barrier resins; nylon polymers; phenolic resins; phenylene-oxide based resins; poly (amide-imide) resins; polyaryl ethers; polyaryl sulfones; polybutadienes; polybutylenes; polycarbonates; polyesters; polyethersulfones; polyethylenes; polyamides; polyimides; polyphenylene sulfides; polypropylenes; polystyrenes; polysulfones; polyurethanes; polyvinyl polymers and resins; silicones; salts of heavy metal cellulose sulfates; gelatin derivatives of which gelatin is the main radical; poly(oxymethylene urea); melamine modified poly(oxymethylene urea); colloidal albumins; hydrolyzed polyvinyl acetate; hydrolysed cellulose esters, e.g., cellulose acetate hydrolysed to acetyl content of 19 to 26 percent; polyacrylamides; imidized polyacrylamides; polyvinyl alcohol; vinyl alcohol polymers containing urethane carboxylic acid groups, e.g., vinyl alcohol cyanoacetate vinyl copolymer; the polymer materials resulting from polymerizing proteins with monomers having a vinyl group; and naturally occurring and synthetic alginates, e.g., salts of water soluble heavy metals such as sodium, potassium and magnesium and combinations and mixtures thereof.

Preferred microcapsules are formed from materials including: starch, gelatin, xanthan gum, poly(oxymethylene urea), and melamine modified poly(oxymethylene urea). Starch, gelatin, and poly(oxymethylene urea) microcapsules are available from a number of sources, e.g., Lipo Technologies of Vadalia, Ohio, Ronald T. Dodge, Co. of Dayton, Ohio, and Minn. Mining and Manufacturing of St. Paul, Minn. Suitable microcapsules may assume various shapes such as spherical, globular, kidney-like, and rice-like. Preferred microcapsules are spherical and have a diameter ranging from about 1 to about 1000 μm, more preferably less than about 100 μm.

Methods of making pressure sensitive microcapsules include, polycondensation, interfacial polymerization, and coacervation/phase separation. These methods are disclosed, for example, in U.S. Pat. Nos. 3,472,675; 3,598,123; and 3,640,629.

Various methods may be used to apply the microcapsules to the floss including, for example, hot melt coating, resin bath coating, spray coating, lick roll coating, and web coating. In addition, the microcapsules may also be incorporated into the floss by various methods including, for example, coating the individual filaments that constitute a strand of floss prior to incorporating the filaments into the strand of floss. A binder, e.g., wax, can optionally be used to coat the floss with microcapsules. These methods are disclosed, for example, in U.S. Pat. No. 5,423,337.

In another embodiment, the erosion control agents may be incorporated in a coating of the dental floss which is deposited on the teeth, such as interproximal spaces, during use. The erosion control agent may be solubilized from the coating and delivered to the oral cavity over a period of time. One coating that may be suitable for use can be formed from a a water insoluble component, such as wax, and a water soluble component, such as gum arabic. Other components such as flavorants, surfactants, emulsifiers, coolants, and water may also be present. In one embodiment, the dental floss coating has a ratio of the water insoluble components (e.g., wax) to the water soluble components (e.g., gum) of between about 1:10, 1:8, 1:6, or 1:4 and about 2:1, 1.5:1, 1:1, or 1:2. The concentrations of water soluble and insoluble components are measured after final processing of the coating, e.g., such as after drying steps during application of the coating to the dental floss. The erosion control agents may also be incorporated directly in the fiber or strands that form all or a portion of the dental floss. The erosion control agents can include any agent which may produce the desired surface protection effects. These agents may also provide desired surface conditioning effects, such as: 1) the effective desorption of portions of undesirable adsorbed pellicle proteins, in particular those associated with tooth stain binding, calculus development and attraction of undesirable microbial species; 2) creating a hydrophilic tooth surface immediately after treatment; and/or 3) maintaining surface conditioning effects and control of pellicle film for extended periods following product use, including post brushing and throughout more extended periods. The effect of creating an increased hydrophilic surface can be measured in terms of a relative decrease in water contact angles. The hydrophilic surface may be maintained on the tooth surface, or a portion thereof, for an extended period after using the product, e.g., flossing.

The polymeric mineral surface active agents can include any agent which will have a strong affinity for enamel surface, deposit a polymer layer or coating on the enamel surface and produce the desired surface protection effects. Some examples of such polymers are polyelectrolytes such as condensed phosphorylated polymers; polyphosphonates; copolymers of phosphate- or phosphonate-containing monomers or polymers with other monomers such as ethylenically unsaturated monomers and amino acids or with other polymers such as proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), poly(propylene glycol), poly(vinyl acetate) and poly(vinyl benzyl chloride); polycarboxylates and carboxy-substituted polymers; and mixtures thereof. Some polymeric mineral surface active agents can include the carboxy-substituted alcohol polymers described in U.S. Pat. Nos. 5,292,501; 5,213,789, 5,093,170; 5,009,882; and 4,939,284; all to Degenhardt et al. and the diphosphonate-derivatized polymers in U.S. Pat. No. 5,011,913 to Benedict et al. A preferred polymer is diphosphonate modified polyacrylic acid. Polymers with activity should have sufficient surface binding propensity to desorb pellicle proteins and remain affixed to enamel surfaces. For tooth surfaces, polymers with end or side chain phosphate or phosphonate functions may both be preferred although other polymers with mineral binding activity may prove effective depending upon adsorption affinity.

Some phosphonate-containing polymers such as shown below are described in U.S. Pat. No. 5,980,776 to Zakikhani, et al.

1. Co-Polymer of Acrylic Acid and Diphosphonic Acid with Structure:

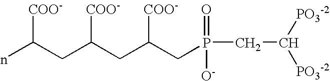

2. Co-Polymer of Acrylic Acid and Vinylphosphonic Acid with Structure:

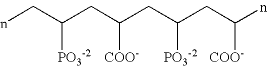

3. Co-Polymer of Methacrylic Acid and Vinlyphosphonic Acid with Structure:

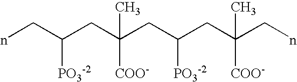

4. Co-Polymer of Acrylic Acid and Vinlydiphosphonic Acid with Structure:

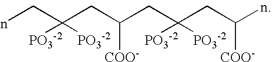

A polymeric mineral surface active agent should be stable with other components of the oral care composition such as ionic fluoride and metal ions and will not hydrolyze in high water content formulations. If the polymeric mineral surface active agent does not have these stability properties, one option is a dual phase formulation with the polymeric mineral surface active agent separated from the fluoride or other incompatible component, such as providing two different types of microcapsules on a dental floss where one microcapsule contains the erosion control agent and the other microcapsule contains the other incompatible agent. Another option is to formulate a non-aqueous, essentially non-aqueous or limited water compositions to minimize reaction between the polymeric mineral surface active agent and other components.

One polymeric mineral surface active agent is a polyphosphate. A polyphosphate is generally understood to consist of two or more phosphate molecules arranged primarily in a linear configuration, although some cyclic derivatives may be present. Although pyrophosphates are technically polyphosphates, the polyphosphates desired are those having around three or more phosphate molecules so that surface adsorption at effective concentrations produces sufficient non-bound phosphate functions, which enhance the anionic surface charge as well as hydrophilic character of the surfaces. The pyrophosphates are discussed separately under additional anticalculus agents. The inorganic polyphosphate salts desired include tripolyphosphate, tetrapolyphosphate and hexametaphosphate, among others. Polyphosphates larger than tetrapolyphosphate usually occur as amorphous glassy materials. Preferred in this invention are the linear “glassy” polyphosphates having the formula:

XO(XPO₃)ₙX

wherein X is sodium or potassium and n averages from about 3 to about 125. Preferred polyphosphates are those having n averaging from about 6 to about 21, such as those manufactured by FMC Corporation and commercially known as Sodaphos (n≈6), Hexaphos (n≈13), and Glass H (n≈21). A particularly preferred polyphosphate has n averaging about 21 such as Glass H. These polyphosphates may be used alone or in a combination thereof.

The amount of polymeric mineral surface agent required is an effective amount to provide the protection from erosion due to acid or abrasive challenges. The protection may last for at least about an hour after use of the composition. An effective amount of a polymeric mineral surface active agent may be from about 1%, 5%, 10%, 20%, 30%, 40%, 50% and or less than about 75%, 70%, 60%, or 50% by weight of the composition containing the erosion control agent. In some embodiments, the dental floss contains between about 2, 4, 6, 8, 10, 15, 20, 25, 30, 40 and/or less than about 100, 90, 80, 70, 60, 50, or 40 micrograms of the erosion control agent per mm of length of the dental floss.

The metal ions suitable for use in the present invention have strong affinity for enamel surface and can include stannous, copper and zinc ions. These ions may provide surface protection effects by reacting with tooth surface ions and/or other components of the composition to produce highly insoluble compounds on the surface. Additionally, these metal ions undergo oxidation and hydrolysis under salivary pH conditions and can produce insoluble deposits on tooth surfaces.

A dental floss may comprise a metal ion source as the erosion control agent that provides stannous ions, zinc ions, copper ions, or mixtures thereof. The metal ion source can be a soluble or a sparingly soluble compound of stannous, zinc, or copper with inorganic or organic counter ions. Examples include the fluoride, chloride, chlorofluoride, acetate, hexafluorozirconate, sulfate, tartrate, gluconate, citrate, malate, glycinate, pyrophosphate, metaphosphate, oxalate, phosphate, carbonate salts and oxides of stannous, zinc, and copper. An effective amount may be from at least about 500 ppm to about 20,000 ppm metal ion of the composition containing the metal ion source, or from about 2,000 ppm to about 15,000 ppm. More preferably, metal ions are present in an amount from about 3,000 ppm to about 13,000 ppm and even more preferably from about 5,000 ppm to about 10,000 ppm. This is the total amount of metal ions (stannous, zinc, copper and mixtures thereof) that is present in the compositions, such as the dental floss coating, for delivery to a tooth surface.

The preferred stannous salts are stannous fluoride and stannous chloride dihydrate. Other suitable stannous salts can include stannous acetate, stannous tartrate and sodium stannous citrate. Examples of suitable zinc ion sources are zinc oxide, zinc sulfate, zinc chloride, zinc citrate, zinc lactate, zinc gluconate, zinc malate, zinc tartrate, zinc carbonate, zinc phosphate, and other salts listed in U.S. Pat. No. 4,022,880. Zinc citrate and zinc lactate are particularly preferred. Examples of suitable copper ion sources are listed in U.S. Pat. No. 5,534,243. The combined metal ion source(s) will be present in an amount of from about 0.1% to about 11%, by weight of the composition containing the metal ion source. The metal ion sources may be present in an amount of from about 0.5 to about 7%, or from about 1% to about 5%. The stannous salts may be present in an amount of from about 0.1 to about 7%, or from about 1% to about 5%, or from about 1.5% to about 3% by weight of the composition containing the stannous salt. The amount of zinc or copper salts which may be used is from about 0.01 to about 5%, or from about 0.05 to about 4% or from about 0.1 to about 3.0%. Other ingredients which may be added to compositions used with the dental flosses of the present invention include, water, buffering agents, thickening agents, adhesive agents, abrasives, surfactants, coloring agents, flavorants, sweetening agents, and antimicrobial agents.

Some antimicrobial agents that may be suitable for use include water insoluble non-cationic antimicrobial agents such as halogenated diphenyl ethers, phenolic compounds including phenol and its homologs, mono and poly-alkyl and aromatic halophenols, resorcinol and its derivatives, bisphenolic compounds and halogenated salicylanilides, benzoic esters, and halogenated carbanilides. The water soluble antimicrobials include quaternary ammonium salts and bis-biquanide salts, among others. Triclosan monophosphate is an additional water soluble antimicrobial agent. The quaternary ammonium agents include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethylstearyl ammonium chloride, cetyl pyridinium chloride, quaternized 5-amino-1,3-bis(2-ethyl-hexyl)-5-methyl hexahydropyrimidine, benzalkonium chloride, benzethonium chloride and methyl benzethonium chloride are exemplary of typical quaternary ammonium antibacterial agents. Other compounds are bis[4-(R-amino)-1-pyridinium]alkanes as disclosed in U.S. Pat. No. 4,206,215, issued Jun. 3, 1980, to Bailey. Other antimicrobials such as copper bisglycinate, copper glysinate, zinc citrate, and zinc lactate may also be included. Also useful are enzymes, including endoglycosidase, papain, dextranase, mutanase, and mixtures thereof. Such agents are disclosed in U.S. Pat. No. 2,946,725, Jul. 26, 1960, to Norris et al. and in U.S. Pat. No. 4,051,234, Sep. 27, 1977 to Gieske et al. Specific antimicrobial agents include chlorhexidine, triclosan, triclosan monophosphate, and flavor oils such as thymol. Triclosan and other agents of this type are disclosed in Parran, Jr. et al., U.S. Pat. No. 5,015,466, issued May 14, 1991, and U.S. Pat. No. 4,894,220, Jan. 16, 1990 to Nabi et al. The quaternary ammonium agents, stannous salts, and substituted guanidines are preferably present in an oral composition separate from the polymeric mineral surface active agent. These agents may be present at levels of from about 0.01% to about 1.5%, by weight of the composition.

The method of use for providing immediate and sustained protection against dental erosion herein comprises contacting a subject's dental enamel surfaces in the mouth with a dental floss. A dentifrice, rinse, or other oral care composition, such as described in U.S. Pat. No. 6,685,920, comprising a polymeric surface active agent, metal ions selected from stannous, zinc, and copper, and combinations thereof, may be used in combination with the dental flosses of the present invention. Further, kits containing such oral care compositions and dental flosses may be provided. In one embodiment, the dental floss is used on enough teeth to deposit at least about 50, 75, 100, 200, 300, 400, 500, 1000, 1,500, 2,000, 5,000 and/or less than about 20,000, 15,000, 10,000, 9,000, 8,000, or 7,000 micrograms of the composition containing the erosion control agent within the oral cavity. The desired amount of deposition may be varied based upon the concentration of the erosion control agent in the composition that is deposited. The composition containing the erosion control agent may be deposited at least one intersitial space between of the incisors and/or the canines of the mandibular and/or maxillary arches. In other embodiments, the composition is deposited at least 2, 3, 4, 5, or 6 and/or less 10, 8, or 6 of the intersitial spaces between of the incisors and/or the canines of the mandibular and/or maxillary arches to achieve the desired deposition.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dental floss, comprising:
at least one fiber; and
a polyphosphate salt.

2. The dental floss of claim 1, wherein the polyphosphate salt has a formula of:
XO(XPO₃)ₙX
and wherein X is sodium or potassium and n averages from about 3 to about 125.

3. The dental floss of claim 1, wherein the polyphosphate salt is disposed within a plurality of microcapsules.
